(19) United States
(12) Patent Application Publication
(10) Pub. No.: US 2015/0208708 A1
(43) Pub. Date: Jul. 30, 2015

(54) OLEOYLETHANOLAMIDE COMPOSITIONS

(71) Applicant: NESTEC S.A., Vevey (CH)

(72) Inventors: Laurent Sagalowicz, Blonay (CH); Ivan Filipi, St. Louis, MO (US); Jean-Marc Juan, Le Landeron (CH); Matthieu Pouzot, Lausanne (CH); Joeska Husny, Bern (CH)

(73) Assignee: NESTEC S.A., Vevey (CH)

(21) Appl. No.: 14/423,875

(22) PCT Filed: Sep. 9, 2013
(86) PCT No.: PCT/EP13/68543

§ 371 Date: Feb. 25, 2015

(30) Foreign Application Priority Data
Sep. 7, 2012 (EP) 12183518.5

(51) Int. Cl. A23L 1/29; A23K 1/16; A61K 9/14; A23L 2/52; A61K 31/164; A61K 9/107

(52) U.S. Cl. CPC A23L 1/293; A61K 31/164; A61K 9/107; A61K 9/14; A23L 2/52; A23K 1/164; A23V 2002/00

(57) ABSTRACT

The present invention relates to the field of N-acyl ethanolamides such as a oleoylethanolamide. In particular the invention relates to flowable emulsions comprising oleoylethanolamide; compositions comprising these emulsions; and the possible uses of such compositions. A further aspect of the invention is a free-flowing powder comprising oleoylethanolamide. The free-flowing powder may, for example, be obtained by drying the non-crystalline emulsions comprising oleoylethanolamide. Further aspects of the invention include the use of the free-flowing powder comprising oleoylethanolamide; and compositions comprising such powders.

The present invention relates to the field of N-acyl ethanolamides such as oleoylethanolamide. In particular the invention relates to flowable emulsions comprising oleoylethanolamide; compositions comprising these emulsions; and the possible uses of such compositions. A further aspect of the invention is a free-flowing powder comprising oleoylethanolamide. The free-flowing powder may, for example, be obtained by drying the non-crystalline emulsions comprising oleoylethanolamide. Further aspects of the invention include the use of the free-flowing powder comprising oleoylethanolamide; and compositions comprising such powders.

Oleoylethanolamide (OEA) is a naturally occurring lipophilic bioactive compound, a member of the N-acylethanolamide family with the IUPAC name (Z)-N-(2-hydroxyethyl)octadec-9-enamide. Oleoylethanolamide is known to control food intake and induce satiety (P. Dipasquale et al., Drug Discovery Today: Disease Mechanisms, 7, e169-e174 (2010)). Oleoylethanolamide has also been shown to influence insulin receptor signalling (M. M. de Ubago et al., Biochimica et Biophysica Acta-Molecular and Cell Biology of Lipids, 1791, 740-745 (2009)). In addition it enhances memory consolidation (P. Campolongo et al., Proceedings of the National Academy of Sciences of the United States of America, 106, 8027-8031 (2009)) and affects the sleep-waking cycle (E. K. Soria-GÓmez et al., Pharmacological Research, 61, 379-384 (2010)). It would be desirable to be able to formulate a range of compositions containing oleoylethanolamide, for example food compositions to help control body weight. Unfortunately oleoylethanolamide presents significant technical challenges for incorporation into other compositions.

Active compounds generally need to be added to compositions such as food products in small, accurately dosed quantities. For this to be possible, the active compound is typically added in the form of a free-flowing liquid or powder. Commercially produced oleoylethanolamide melts over a temperature range between 50 and 70° C. and so would need to be held above 70° C. in order to dose it as a flowable liquid. This has two significant drawbacks. Dosing a molten material requires non-standard storage and dosing equipment but it also risks the oleoylethanolamide decomposing. The unsaturated fatty acid chain in the oleoylethanolamide molecule makes oleoylethanolamide susceptible to oxidation, and so storage at elevated temperatures for prolonged periods, for example in a dosing reservoir, is problematic. Oleoylethanolamide is generally supplied as pellets or flakes. It is a sticky material which compacts if an attempt is made to grind it to a powder. If oleoylethanolamide is melted and then spray-chilled the resulting powder rapidly cakes on storage under typical ambient conditions and is not free-flowing. Essentially, oleoylethanolamide has too high a melting point to be conveniently dosed as a liquid, but too low a melting point to form a stable free-flowing powder, especially in the case of commercially available oleoylethanolamide material which may contain other minor components which lower its melting onset temperature and widen its melting range. Melting oleoylethanolamide together with a higher melting fat and then spray-chilling could produce a stable powder, but it has the disadvantage that the higher melting fat may interfere with the properties of the composition into which it is added. For example, high melting fats alter the organoleptic properties of food materials.

Spray drying is a well known method for forming a powder, for example where an ingredient is first dissolved in water and then spray dried to a powder. However, when oleoylethanolamide is mixed with water it forms a liquid crystalline phase (EP2107097) which is very viscous and difficult to disperse. The high viscosity and lack of homogeneity of this liquid crystalline material makes it unsuitable both for dosing applications and as a feedstock for spray drying.

Oleoylethanolamide can be dissolved in water using co-solvents. For example US52003/0018081 discloses dissolving oleoylethanolamide at a level of 2% in a mixture of 5% Tween 80, 5% propylene glycol and 90% saline for the purpose of an intraperitoneal injection of oleoylethanolamide. However, this specialist solvent mix has a number of disadvantages for dosing oleoylethanolamide or for spray drying. Tween 80 and propylene glycol will be incorporated into any final product which may not be desired, especially as the quantity of these materials used to solubilise the oleoylethanolamide exceeds the amount of oleoylethanolamide. Propylene glycol for example is not suitable for use in foods for cats because of a species-specific reaction in the cat's body.

Hence, there is a clear need in the industry to find alternative physical forms for oleoylethanolamide which would, for example, allow oleoylethanolamide to be conveniently and accurately dosed into other materials.

The object of the present invention is to improve the state of the art and to provide an improved solution to overcome at least some of the inconveniences described above or at least to provide a useful alternative. Any reference to prior art documents in this specification is not to be considered an admission that such prior art is widely known or forms part of the common general knowledge in the field. As used in this specification, the words “comprises”, “comprising”, and similar words, are not to be interpreted in an exclusive or exhaustive sense. In other words, they are intended to mean “including, but not limited to”.

The object of the present invention is achieved by the subject matter of the independent claims. The dependent claims further develop the idea of the present invention.

Accordingly, the present invention provides in a first aspect a flowable emulsion comprising oleoylethanolamide, oil, water and an emulsifier. The inventors initially attempted to form an emulsion by combining oleoylethanolamide, water and an emulsifier. However, the resulting mixture was inhomogeneous, the oleoylethanolamide and water forming a viscous liquid-crystal phase which the emulsifier was not able to disperse. However, the inventors were surprised to find that, when they added oil to the oleoylethanolamide, a low viscosity liquid was formed which could be dispersed as a stable homogeneous emulsion with the addition of an emulsifier.

In a second aspect, the invention provides a free-flowing powder comprising oleoylethanolamide. The inventors were surprised to find that, when the stable io homogeneous emulsion of oleoylethanolamide, oil, water and an emulsifier was spray dried, it formed a free-flowing powder. Accordingly, the invention also provides a method for producing a free-flowing powder containing oleoylethanolamide.

The invention further provides compositions comprising the flowable emulsion comprising oleoylethanolamide, oil, water and an emulsifier; as well as compositions comprising the free-flowing powder comprising oleoylethanolamide. The flowable nature of both the emulsion and the free-flowing powder allow them to be readily added to other compositions.

A further aspect of the invention is a flowable emulsion comprising oleoylethanolamide, oil, water and an emulsifier for use in the treatment or prevention of obesity or being overweight. A still further aspect of the invention is a free-flowing powder comprising oleoylethanolamide for use in the treatment or prevention of obesity or being overweight. The invention also provides for the non-therapeutic use of the flowable emulsion comprising oleoylethanolamide, oil, water and an emulsifier; as well as for the non-therapeutic use of the free flowing powder comprising oleoylethanolamide. Such non-therapeutic uses may be to increase satiety, or to improve memory consolidation.

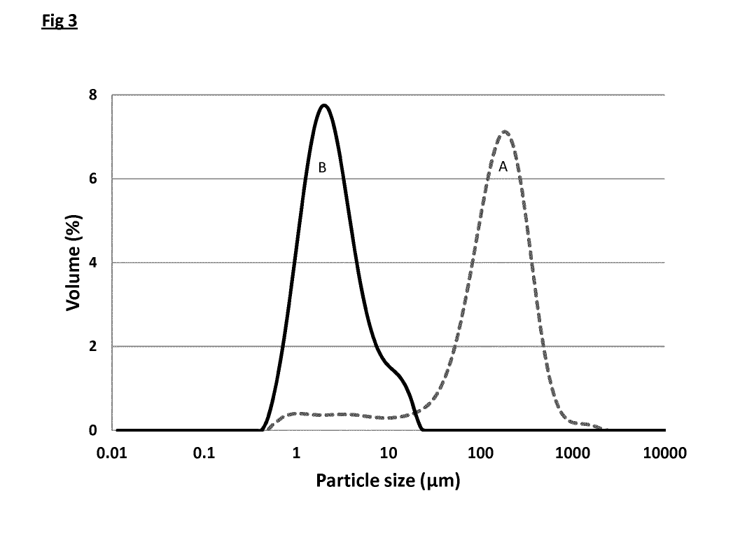

FIG. 3. shows the effect of gravity after inverting test tubes containing a mixture of oleoylethanolamide and water without oil (A, dashed line) and with oil (B, solid line) at 55° C.

FIG. 2 shows dispersed oleoylethanolamide in water without oil (A) and with oil (B) in presence of emulsifier (whey protein isolate) at 60° C.

FIG. 3 shows the particle size distribution of oleoylethanolamide dispersed in water in the presence of emulsifier (whey protein isolate) at 40° C. without oil (A) and with oil (B).

Consequently the present invention relates in part to a flowable emulsion comprising oleoylethanolamide, oil, water and an emulsifier. An emulsion is a mixture of two or more liquids that are normally immiscible. In an emulsion, one liquid (the dispersed phase) is dispersed in the other (the continuous phase). Emulsions are distinct from other colloids having a liquid continuous phase such as foams and sols. In a foam the dispersed phase is a gas; in a sol the dispersed phase is a solid. Examples of sols include pigmented ink and blood. Two liquids can form different types of emulsion. As an example, oil and water can form an oil-in-water emulsion or they can form a water-in-oil emulsion. Multiple emulsions are also possible, namely water-in-oil-in-water or oil-in-water-in-oil. In the scope of the present invention, an emulsion refers to a homogeneous dispersion. The emulsion of the invention has a dispersed phase which is homogeneously dispersed through the continuous phase with a droplet size having a D[4,3] (sometimes called De Brouckere mean diameter) less than 100 μm. In the present invention, the term flowable emulsion means that the emulsion has a dynamic viscosity of less than 1 Pa·s at 60° C. For example the emulsion of the invention may have a dynamic viscosity less than 0.5 Pa·s, for further example less than 0.1 Pa·s at 60° C.

Oleoylethanolamide may be present in the emulsion of the present invention at a level of at least 0.5 wt. %, for example at least 5 wt. %, for further example at least 10 wt. %, for further example at least 15 wt. %.

The term oils in the scope of the present invention means lipid materials with an HLB number lower than 3.5, for example an HLB number lower than 3. The term “HLB number” refers to the hydrophilic-lipophilic balance. The HLB number is determined by the method described by Griffin (W. C. Griffin, Journal of the Society of Cosmetic Chemists, 1, 311 (1949)) and (W. C. Griffin, Journal of the Society of Cosmetic Chemists 5, 249 (1954)). The oils may comprise triglycerides, diglycerides, free fatty acids, fatty acid derivatives or mixtures of these. The oils may be added to the emulsion as such, or be comprised within another ingredient, for example the oils may be present as other minor components within commercial OEA materials. The oils may be liquid at the processing temperature, for example liquid above 85° C.; for further example liquid above 55° C.; or for further example liquid above 25° C. In the present invention the oil may be fish oil, animal oil or vegetable oil. For example, the animal oil may be extracted from the rendered tissue of livestock animals like pigs, chickens and cows, or be dairy oils such as butter oil. The vegetable oil may be selected from the group consisting sunflower oil, rapeseed oil, cottonseed oil, coconut oil, olive oil, palm oil, soybean oil, peanut oil, palm kernel oil, corn oil, grape seed oil, hazelnut oil, sesame oil and mixtures of these.

The emulsion of the present invention may be non-crystalline. Such emulsions provide flowability and stability over a wide range of oleoylethanolamide content. The term non-crystalline refers to the absence of crystals, including the absence of liquid crystals. Liquid crystals are a state of matter that has properties between those of a conventional liquid and those of a solid crystal. Liquid crystals can have positional order (molecules arranged in any sort of ordered lattice) and/or orientational order (molecules mostly pointing in the same direction). The order can be either short-range or long-range. Some emulsions can exhibit a small degree of long-range order, but in the scope of the present invention liquid crystalline phases are selected from the group consisting of the reverted micellar cubic phase (space group Q227 and symmetry fd-3m); the lamellar liquid crystalline phase (Lα); the reverse hexagonal phase (H₂); and the inverse bicontinuous cubic phase such as the primitive type (space group Q229 and symmetry: Im-3m), the gyroid type (space group Q230 and symmetry Ia-3d) and the diamond type (space group Q₂₂₄, symmetry Pn-3m). Crystals can be detected by any of the methods well known in the art, for example X-ray diffraction, Small-Angle X-ray Scattering (SAXS) measurements, cross polarized io microscopy or Differential Scanning calorimetry (DSC). In the scope of the current invention, non-crystalline means that less than 5% of the non-water components of the emulsion by weight may be crystalline, for example less than 1% of the non-water components of the emulsion by weight may be crystalline, for example none of the non-water components of the emulsion may be crystalline.

An emulsifier is a surface-active agent that facilitates the formation and stability of an emulsion. The emulsifier of the present invention may be any of the many emulsifying agents known to be capable of stabilizing an oil-in-water emulsion. The emulsifier may be selected from the group consisting of phospholipids, for example lecithin; lysophospholipids; glycolipids; monoglyceride derivatives, for example acetic acid esters of monoglycerides, lactic acid esters of monoglycerides, citric acid esters of monoglycerides, succinic acid esters of monoglycerides, diacetyl tartaric acid esters of monoglycerides; fatty acid esters, for example polyglycerol esters of fatty acids, sorbitan esters of fatty acids, propylene glycol esters of fatty acids, sucrose esters of fatty acids; emulsifying block-copolymers; saponins; surface active particles such as colloidal particles from zein; sodium stearoyl lactylate; calcium stearoyl lactylate; proteins, for example whey protein, whey protein isolate, casein, sodium caseinate; surface active hydrocolloids, for example modified starch, acacia gum; peptides; or mixtures of these. Some materials may act either as an emulsifier or be themselves emulsified as an oil, depending on the other ingredients, for example free fatty acids and fatty acid derivatives. The emulsifier of the invention may have an HLB number greater than 4, for example greater than 6, for further example greater than 8. It should be understood that where an HLB number cannot be determined according to the method of Griffin (e.g. for high molecular weight emulsifiers) the emulsifier of the invention may have the equivalent hydrophile/lipophile character as an emulsifier with an HLB number greater than 4, for example greater than 6, for further example greater than 8.

The emulsifier of the present invention may be naturally occurring in another food material such as egg yolk (in which the main emulsifying agent is lecithin) or mustard (where a variety of chemicals in the mucilage surrounding the seed hull act as emulsifiers). Both the oil and the emulsifier may be comprised within another ingredient. For example enzymatically hydrolyzed meat or fish (sometimes called “animal digest” or simply “digest”) is used in the formulation of some pet foods. This typically contains emulsifiers in the form of proteins and peptides, as well as oils. The oil and emulsifier in the emulsion of the current invention may be added in the form of enzymatically hydrolyzed meat or fish.

The emulsion of the present invention may be food grade. All components of the emulsion may be suitable for use in a material to be consumed as food. Ingredient legislation varies around the world, but preferably all components of the emulsion may be approved as ingredients for food, including pet-food. Many people prefer not to eat non-aqueous solvents, and religious dietary laws may prohibit the consumption of ethanol. Although some non-aqueous solvents may be present in low amounts in food, for example propylene glycol as a carrier for flavours, larger amounts are generally undesirable. The emulsion of the present invention may contain less than 3 wt. % of non-aqueous solvents miscible in water, for example less than 1 wt. %, for further example less than 0.5 wt. %. The emulsion of the present invention may be free from non-aqueous solvents miscible in water.

The emulsion of the present invention may have a ratio (w/w) of oil to oleoylethanolamide of between 0.02:1 and 2:1, for example between 0.1:1 and 1:1. These ratios of oil to oleoylethanolamide provide a stable homogeneous emulsion. The emulsion of the present invention may have a ratio (w/w) of emulsifier to oleoylethanolamide of between 0.2:1 and 2:1, for example between 0.4:1 and 1:1. The emulsifier of the present invention may not be oleoylethanolamide.

The emulsion of the present invention provides a suitable physical form for dosing oleoylethanolamide conveniently and accurately into other materials. A composition comprising the emulsion of the invention may be a food composition, a pharmaceutical composition, a food additive, a drink, a nutritional formulation, a tube feeding formulation or a pet food composition.

Oleoylethanolamide will start to crystallize below 50° C., and so emulsions according to the invention containing more than 5% oleoylethanolamide are most stable above this temperature. The emulsion of the present invention may be at a temperature between 40° C. and 90° C., for example between 55° C. and 85° C. However, as the dilution of the emulsion is increased, the emulsions remain stable on cooling. For example, the emulsion of the invention may be added to another emulsion such as ruminant milk, for example cows' milk. With levels of oleoylethanolamide in the milk of less than 1%, the oleoylethanolamide remains dispersed even when the milk is chilled. The composition comprising the emulsion of the present invention may be milk, for example ruminant milk, soy milk, almond milk, rice milk or peanut milk. The milks may be flavoured and/or coloured.

The emulsion of the present invention may be for use in the treatment or prevention of obesity or being overweight. Being overweight or obese are well-known disorders that represent a significant burden in our society today. Overweight is defined for an adult human as having a Body Mass Index (BMI) between 25 and 30. BMI is calculated as the ratio of weight in kg divided by height in metres, squared. Obesity is a condition in which the natural energy reserve stored in the fatty tissue of animals, in particular humans and other mammals, is increased to a point where it is associated with certain health conditions or increased mortality. Obese is defined for an adult human as having a BMI greater than 30. In recent years, obesity has become the most common nutritional disorder encountered in small animal medicine. Obesity is a serious medical condition that can lead to a variety of related health problems as well as shortened life span. Spaying or neutering of cats and dogs is a major risk factor for a significant decline in energy metabolism with an excessive accumulation of body fat. Obesity in cats and dogs may be determined by measuring body weight and assessing Body Condition Scores (BCS) (Overview of Nutrition: Small Animals>> Merck Veterinary Manual [online] July 2011 [retrieved on Jul. 24, 2012] <URL:www.merckmanuals.com/vet/management_and_nutrition/nutrition_small_animals/overview_of_nutrition_small_animals.html#v4643413/>). On the 9-point BCS scale, in the scope of the current invention, dogs or cats scoring 5 or 7 are considered overweight and 8 or 9 are obese.

The emulsion of the present invention provides oleoylethanolamide in a physical form which is well suited to being incorporated in the normal components of a daily diet and can increase satiety. Healthy people often desire to moderate their food intake to keep a healthy body weight. Owners of healthy pets would like their pet's appetite to be satiated after an appropriate intake of food so that they do not continue to pester for food. The invention provides for the non-therapeutic use of the emulsion of the invention to increase satiety.

Oleoylethanolamide has been shown to enhance memory consolidation (P. Campolongo et al., Proceedings of the National Academy of Sciences of the United States of America, 106, 8027-8031 (2009)), a process whereby memories are stabilized after the initial acquisition. Improvements in memory consolidation are desirable not just people those who have a medical problem with their memory. Non-therapeutic improvements in memory consolidation are those which result in a healthy, normal level of memory consolidation being enhanced. Healthy people may have concerns about their power of recollection, for example if they are studying for an important examination. Owners of healthy pets generally like their pets to have good memories, to be able to learn and remember tricks or daily routines for example. The invention provides for the non-therapeutic use of the emulsion of the invention to improve memory consolidation.

The inventors were surprised to find that, by spray drying an emulsion of oleoylethanolamide, oil, water and an emulsifier they were able to form a free-flowing powder. Accordingly, the invention provides a free-flowing powder comprising oleoylethanolamide. When flowable powders are poured onto a horizontal surface, a conical pile will form. The internal angle between the surface of the pile and the horizontal surface is known as the angle of repose. The critical angle of repose of a powder is the steepest angle of descent of the slope relative to the horizontal plane when material on the slope face is on the verge of sliding. This angle is in the range 0° to 90°. Free flowing powders have a low critical angle of repose as the powder readily flows outwards resulting in a flatter pile. In the current invention, free-flowing powder means that the powder has a critical angle of repose less than 55°. Preferably the free-flowing powder has a critical angle of repose less than 50°. In contrast, the solid forms of oleoylethanolamide known prior to the current invention have wax-like properties and do not flow at all. It is not possible to measure a critical angle of repose for such materials.

Where powder ingredients are used in the manufacture of products, the flow of the powder is a critical part of dosing and dictates the quality of the final product in terms of its weight and content uniformity. A free-flowing powder also enhances manufacturing efficiency, for example allowing a more rapid production rate. It is therefore an advantage to be able to provide a free-flowing powder comprising oleoylethanolamide. The free-flowing powder may comprise oleoylethanolamide, oil and an emulsifier. The invention further provides a free-flowing powder obtainable, for example obtained, by drying the emulsion of the invention. The drying method used to obtain the free-flowing powder may be selected from the group consisting of freeze drying, vacuum drying, spray drying and combinations of these.

The powder of the present invention may have a ratio (w/w) of oil to oleoylethanolamide of between 0.02:1 and 2:1, for example between 0.1:1 and 1:1. These ratios of oil to oleoylethanolamide provide a stable free-flowing powder. The powder of the present invention may have a ratio (w/w) of emulsifier to oleoylethanolamide of between 0.2:1 and 2:1, for example between 0.4:1 and 1:1. The powder of the present invention may contain at least 20% oleoylethanolamide, for example at least 40% oleoylethanolamide or at least 60% oleoylethanolamide. It is beneficial to be able to provide a free-flowing powder with a high content of oleoylethanolamide.

The powder of the present invention may be food grade. All components of the powder may be suitable for use in a material to be consumed as food. Ingredient legislation varies around the world, but preferably all components of the powder may be approved as ingredients for food, including pet-food.

The free-flowing powder of the invention may further comprise anticaking (free-flow) agents. Most commercial powders have agents to improve flow and reduce inter-particle interactions. Anticaking agents used in the invention may be selected from the group comprising tricalcium phosphate, powdered cellulose, sodium bicarbonate, sodium ferrocyanide, potassium ferrocyanide, calcium ferrocyanide, bone phosphate, sodium silicate, silicon dioxide, calcium silicate, magnesium trisilicate, talcum powder, sodium aluminosilicate, potassium aluminium silicate, calcium aluminosilicate, bentonite, aluminium silicate, stearic acid, polydimethylsiloxane, amorphous silica, calcium carbonate, magnesium carbonate or mixtures of these. Anticaking agents may for example comprise up to 5% w/w of the free-flowing powder.

One aspect of the invention is a method for producing a free-flowing powder containing oleoylethanolamide comprising the steps of mixing oleoylethanolamide with oil, water and an emulsifier at a temperature between 55 and 85° C. to form an emulsion; and drying the emulsion to form a powder. Spray drying the emulsion is particularly effective at producing a free-flowing powder; the dispersed emulsified droplets of oleoylethanolamide and oil becoming well separated fine particles with regular shapes as they pass through a spray drying tower. Accordingly the method of the invention may dry the emulsion using spray drying.

Oleoylethanolamide may be present in the emulsion formed in the method of the invention at a level of at least 0.5 wt. %, for example at least 5 wt. %, for further example at least 10 wt. %, for further example at least 15 wt. %.

Being able to accurately dose compositions for treatment or therapy is often critical, and this is possible with free-flowing powders. In addition, such powders can be conveniently administered to subjects in need of therapy. For example, measured amounts can be sprinkled onto food, such as pet food. A free-flowing powder according to the invention may be for use in the treatment or prevention of obesity or being overweight.

Providing a free-flowing powder comprising oleoylethanolamide is convenient for non-therapeutic uses. The powder of the invention may be used non-therapeutically to increase satiety. In a further aspect, the free-flowing powder of the invention may be used non-therapeutically to improve memory consolidation.

The free-flowing powder of the present invention provides a suitable physical form for dosing oleoylethanolamide conveniently and accurately into other materials. A composition comprising the free-flowing powder of the invention may be a food composition, a pharmaceutical composition, a food additive, a powdered composition to be reconstituted in milk or water, a nutritional formulation or a pet food composition. The free-flowing powder may be added into food during preparation, as a cooking ingredient for example. The free-flowing powder of the invention may also be added to food just before consumption. For example, the powder could be provided with a measuring scoop to allow an appropriate dose of the powder to sprinkled over the food, or it could be packed into sachets in pre-measured amounts. This is advantageous when different consumers would like different quantities of the oleoylethanolamide powder. For example, a parent might want to add oleoylethanolamide powder to their own food to enhance satiety and so reduce the amount they eat, but might not want to add the powder to their child's food, who perhaps needs to be encouraged to eat. With dry food, for example kibbled pet food, keeping the powder separate until just before consumption can also help avoid problems of the powder settling in the pack and becoming unevenly distributed. The free-flowing powder of the invention may be in combination with a pet food product wherein the free-flowing powder is packed in single-portion packs to be added to a serving of the pet food product.

Those skilled in the art will understand that they can freely combine all features of the present invention disclosed herein. In particular, features described for the products of the present invention may be combined with the method of the present invention and vice versa. Further, features described for different embodiments of the present invention may be combined. Where known equivalents exist to specific features, such equivalents are incorporated as if specifically referred to in this specification. Further advantages and features of the present invention are apparent from the figures and non-limiting examples.

### EXAMPLES

### Example 1

### Addition of Water to Oleoylethanolamide

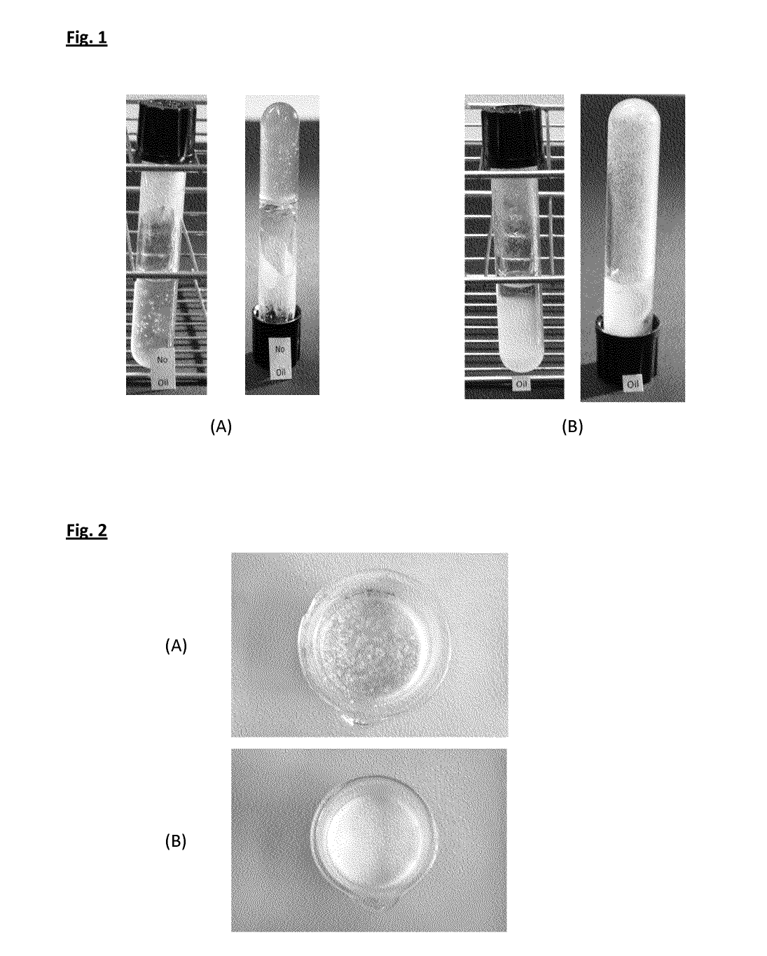

A mixture of 20% water and 80% oleoylethanolamide (Stepan Co.) was prepared at 55° C.—Mix A. The mixture was found to be non-flowable, as a highly viscous liquid crystalline phase formed. Surprisingly, the inventors found that by adding sunflower oil, the mixture became much less viscous; Mix B—72% oleoylethanolamide, 8% sunflower oil and 20% water. The effect of gravity at 55° C. for the two mixtures is shown in FIG. 1. When the test-tube containing Mix A is inverted the mixture does not flow; whereas Mix B, containing oil, readily flows down the test tube.

Mix B is not an emulsion, it is an example of a reduced viscosity material which may form the dispersed phase in an emulsion according to the invention.

### Example 2

### Emulsion with Oleoylethanolamide, Oil and Emulsifier

Whey protein isolate (Prolacta® 84 from Lactalis Ingredients) was dispersed in water and heated to 70° C. Oleoylethanolamide was heated separately at 70° C. The water containing whey protein isolate was added to the oleoylethanolamide and homogenized in a kitchen mixer (Braun 600 Watt turbo) to form a final mixture of 12% OEA, 4% whey protein isolate and 84% water. A stable homogeneous emulsion did not form; the mixture contained lumps even after extended stirring and had a gritty “cheese-curd” like texture. It would not be possible to spray dry such a material.

A second mixture was prepared in the same way, but this time with the addition of sunflower oil to the oleoylethanolamide before it was combined with the whey protein isolate and water. The final mixture contained 9% DEA, 3% sunflower oil, 4% whey protein isolate and 84% water. This mixture formed a homogenous, stable emulsion. FIG. 2 shows photographs of the two mixtures, without oil (A) and with oil (B), demonstrating that oil is essential to obtain a stable homogeneous dispersion of oleoylethanolamide.

The size distributions of the dispersed phases in the two mixtures were analyzed by light scattering using a Malvern™ Mastersizer 2000 fitted with a Hydro 2000 G dispersion unit (stirrer 500 rpm and pump 1500). Hot samples (40-50° C.) were added to water at 25° C. in the dispersion unit. The size distribution of the two samples is shown in FIG. 3. The mixture with oil had a much smaller “particle” size for the dispersed phase, with a D[4,3] of 3.143 μm, while the mixture without oil had a D[4,3] of 176.630 μm.

Viscosity was measured according to the following procedure using a Physica MCR 501 rheometer from Anton Paar. The sample was filled into a Couette device. A thin layer (1±0.05 mm) of a very low viscosity mineral oil was used to cover the sample in order to suppress evaporation or film formation at the contact of the emulsion and air interface. The mixture with oil had a dynamic viscosity of 2±0.2 mPa·s when measured at 60° C., in rotational mode. The inhomogeneous nature of the mixture without oil meant that no sensible measurement was possible.

### Example 3

### Emulsion with Oleoylethanolamide and Animal Digest

Dry animal digest (enzymatically hydrolyzed poultry tissues with 65% proteins/peptides, 24% lipids and 11% moisture) was added to oleoylethanolamide and heated at 70° C. until the oleoylethanolamide had completely melted. Water was heated separately at 70° C. and added to the oleoylethanolamide and digest before being mixed for 3 minutes with a kitchen mixer. The final composition was 8.6% OEA, 20% digest and 71.4% water. The mixture formed a homogeneous emulsion.

### Example 4

### Oleoylethanolamide with Whey Protein Iisolate (No Oil)

Water was placed in a Stephan Mixer and heated to 70° C. using indirect heating. Then, whey protein isolate was added and mixed for a few seconds. Molten (75° C.) oleoylethanolamide was added last. The batch size was 18.14 kg, containing 9% OEA, 4% whey protein isolate and 87% water. After 10 minutes mixing at 70° C. no emulsion formed. A gritty, thick, cheese curd-like texture was obtained. The temperature was increased to 82° C. and mixing continued for a further 15 minutes, but the mixture remained unchanged.

This demonstrates that, without the presence of oil, it is not possible to form an emulsion with OEA and an emulsifier. Raising the temperature did not noticeably affect the viscosity or the homogeneity. The thick, inhomogeneous mixture obtained was unsuitable for spray drying.

### Example 5

### Spray Dried Oleoylethanolamide Powder

An emulsion was prepared using a Stephan Mixer. Water was added first and heated to 70° C. using indirect heating. Then, whey protein isolate and oil were added and mixed for a few seconds. Molten (75° C.) oleoylethanolamide was added last. A smooth emulsion formed almost instantly and mixing continued at 70° C. for 10 min. The batch size was 18.14 kg, containing 9% OEA, 4% whey protein isolate, 3% soybean oil and 84% water.

Approximately 9 kg of emulsion was transferred for spray drying to a pilot scale Niro dryer, equipped with a filter mat. Flow rate was 0.25 kg/min. Hollow cone UniJet 2 (Spraying Systems Co.) was used. During the spray drying, the emulsion was kept in the spray drying tank at 70° C. with constant stirring. The resulting spray dried powder was fine, with no visible OEA separation and was free-flowing. An amorphous silica anticaking agent (Syloid® 244) was added at a level of 1.5%. The powder easily passed through a 300 μm sieve. A sample of the emulsion was stored in a refrigerator at 5° C. for 10 days without any separation being visible.

The critical angle of repose was measured as follows. A glass funnel was mounted on a ring stand over a 7 cm radius petri dish, open side up. The tip of the funnel was adjusted to be 12 cm above the petri dish. The spray-dried powder (incorporating anticaking agent) was gently poured through the funnel onto the petri dish to form a cone of powder. Care was taken to avoid vibration. The addition of powder was discontinued when the powder started to flow over the lip of the petri dish. The height of the cone of powder above the lip of the petri dish was measured. The critical angle of repose (a) was calculated as follows.

$$tan α = \frac{height}{petri dish radius}$$

The critical angle of repose was found to be 38°.

### Example 6

### Spray Dried Oleoylethanolamide Powder Containing Digest

An emulsion was prepared using a Stephan Mixer. Water was added first and heated to 70° C. using indirect heating. Then, dry animal digest, followed by whey protein isolate and oil were added and mixed for a few seconds. Molten (75° C.) oleoylethanolamide was added last. A smooth emulsion formed almost instantly and mixing continued at 70° C. for 10 min. The batch size was 20 kg, containing 7.9% OEA, 4.1% whey protein isolate, 1.8% digest, 6.4% soybean oil and 79.8% water.

Approximately 9 kg of emulsion was transferred for spray drying to a pilot scale Niro dryer, equipped with a filter mat. Flow rate was 0.25 kg/min. Hollow cone UniJet 2 (Spraying Systems Co.) was used. During the spray drying, the emulsion was kept in the spray drying tank at 70° C. with constant stirring. The resulting spray dried powder was very fine, with no visible OEA separation. An amorphous silica anticaking agent (Syloid® 244) was added at a level of 1.5%. The powder easily passed through a 300 μm sieve. The powder was free-flowing, having a critical angle of repose of 47°.

1. Flowable emulsion comprising oleoylethanolamide, oil, water and an emulsifier.

2. A flowable emulsion according to claim 1 wherein the emulsion is non-crystalline.

3. A flowable emulsion according to claim 1 wherein the oil and emulsifier are added in the form of enzymatically hydrolyzed meat or fish.

4. A method for the treatment or prevention of obesity or being overweight comprising administering to an individual in need of same a composition comprising flowable emulsion comprising oleo lethanolamide oil water and an emulsifier.

5. A non-therapeutic method to increase satiety comprising administering to an individual in need of same a composition comprising flowable emulsion comprising oleoylethanolamide, oil, water and an emulsifier.

6. A non-therapeutic method to improve memory consolidation comprising administering to an individual in need of same a composition comprising flowable emulsion comprising oleo lethanolamide oil water and an emulsifier.

7. Composition comprising the flowable emulsion of claim 1 wherein the composition is in a form selected from the group consisting of a food composition, a pharmaceutical composition, a food additive, a drink, a nutritional formulation, a tube feeding formulation, and a pet food composition.

8. Free-flowing powder comprising oleoylethanolamide.

9. A free-flowing powder according to claim 8 wherein the free-flowing powder is obtainable by drying an emulsion comprising oleoylethanolamide oil water and an emulsifier.

10. A method for the treatment or prevention of obesity or being overweight comprising administering to an individual in need of same a composition comprising flowable emulsion comprising oleoylethanolamide, oil, water and an emulsifier.

11. A non-therapeutic method to improve memory consolidation comprising administering to an individual in need of same a composition comprising flowable emulsion comprising oleoylethanolamide, oil, water and an emulsifier.

12. A non-therapeutic method to increase satiety comprising administering to an individual in need of same a composition comprising flowable emulsion comprising oleoylethanolamide, oil, water and an emulsifier.

13. Composition comprising the free-flowing powder according to claim 8 wherein the composition is in a form selected from the group consisting of a food composition, a pharmaceutical composition, a food additive, a powdered composition to be reconstituted in milk or water, a nutritional formulation and a pet food composition.

14. A free-flowing powder comprising in combination with a pet food product wherein the free-flowing powder is packed in single-portion packs to be added to a serving of the pet food product.

15. Method for producing a free-flowing powder containing oleoylethanolamide comprising the steps:
mixing oleoylethanolamide with oil, water and an emulsifier at a temperature between 55 and 85° C. to form an emulsion: and
drying the emulsion to form a powder.
